(19) United States
(12) Patent Application Publication
(10) Pub. No.: US 2009/0281604 A1
(43) Pub. Date: Nov. 12, 2009

(54) CONTROLLING A PHOTO-BIOLOGICAL EFFECT WITH LIGHT

(75) Inventors: Dirk Kornelis Gerhardus De Boer, Eindhoven (NL); Lucas Josef Maria Schlangen, Eindhoven (NL); Adrianus Johannes Stephanus Maria De Vaan, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/296,677

(22) PCT Filed: Apr. 3, 2007
(86) PCT No.: PCT/IB07/51180

§ 371 Date: Oct. 10, 2008

(30) Foreign Application Priority Data
Apr. 11, 2006 (EP) 06112471.5

(51) Int. Cl. A61N 5/06

(52) U.S. Cl. 607/88; 345/83

(57) ABSTRACT

A device for generating at least blue light comprises a control circuit (4) which receives a control signal (CS) defining a variation of a spectrum of the blue light to control a photo-biological effect of a vertebrate. Therefore, first blue light (BL1) is generated with a first predominant wavelength (PW1) having a first photo-biological effect, or second blue light (BL2) is generated with both a second predominant wavelength (PW2), being shorter than the first predominant wavelength, and a third predominant wavelength (PW3), being longer than the first predominant wavelength; the second blue light (BL2) has a second photo-biological effect different from the first photo-biological effect, while the first blue light (BL1) and the second blue light (BL2) have substantially identical colors and intensities.

### FIELD OF THE INVENTION

The invention relates to a device for generating at least blue light, a display device comprising pixels for generating the at least blue light, a backlight unit for a display device, and a method of generating at least blue light.

### BACKGROUND OF THE INVENTION

JP2005-063687 discloses that a living body has a timer which defines the circadian rhythm of the body. Sleepiness, alertness and temperature change according to the circadian rhythm. The biorhythm is controlled by the amount of melatonin secretion. It was found that light influences the melatonin secretion. The secretion of melatonin is maximally suppressed by light having a wavelength of 470 nm. JP2005-063687 further discloses a light-emitting device and display device exerting a biological rhythm control by emitting blue light with a wavelength of 445 to 480 nm. The light-emitting device has red LEDs, green LEDs, first blue LEDs, and second blue LEDs. The first blue LEDs emit light with a peak at 470 nm, the second blue LEDs emit light with a peak at a shorter wavelength than the first blue LEDs. The melatonin restriction effect is controlled by selecting between the first blue LEDs and the second blue LEDs.

It is a drawback of this light emitting device that the color and/or intensity of the light varies when the melatonin suppression effect is changed.

### SUMMARY OF THE INVENTION

It is an object of the invention to provide a device for varying light to obtain a different effect on the melatonin suppression but substantially the same color and intensity.

A first aspect of the invention provides a device for generating at least blue light as claimed in claim 1. A second aspect of the invention provides a display device comprising pixels as claimed in claim 10. A third aspect of the invention provides a backlight unit for a display device as claimed in claim 18. A fourth aspect of the invention provides a method of generating at least blue light as claimed in claim 19. Advantageous embodiments are defined in the dependent claims.

A device in accordance with the first aspect of the invention generates at least blue light and has a control circuit which varies the spectrum of the blue light dependent on a control signal to control a photo-biological effect of a vertebrate. The photo-biological effect may be a melatonin suppression effect and/or a biological stimulating/alerting effect on subjects without any measurable effect on melatonin levels. The spectrum of the blue light can be varied by generating first blue light with a first predominant wavelength having a first photo-biological effect, and/or by generating a second blue light with both a second predominant wavelength, being shorter than the first predominant wavelength, and a third predominant wavelength, being longer than the first predominant wavelength. The second blue light has a second photo-biological effect smaller than the first photo-biological effect, while the first blue light and the second blue light have substantially identical colors and perceived intensities. Switching between the first and the second blue light may be instantaneous, but is preferably a slow transition which may take hours, such that the light slowly changes from the first to the second photo-biological effect, or the other way around.

The first blue light has a predominant first wavelength in between the predominant second and third wavelengths of the second blue light. By using this single blue light source or the two blue light sources in combination with each other, it is possible to obtain substantially the same color and intensity of the blue light but different photo-biological effects.

It is especially important that the color and intensity of the blue light do not change substantially in applications where the blue light is used as one of the primaries of a color display. This allows to produce the same visual image but with different photo-biological effects. By substantially the same color and intensity is meant that the viewer does not observe a change of the color and/or intensity irrespective of whether the first or the second blue light is used for the blue primary. In a backlight unit, the blue light may be produced by one or more lamps or LEDs. In a CRT or PDP, the blue light may be produced by phosphor dots or stripes. The skilled person readily understands that for creating a substantially identical color and intensity, there are many possibilities to select the two predominant wavelengths of the second blue light and the intensity thereof. From the fact that a different photo-biological effect has to be reached it is clear that the skilled person, knowing the photo-biological curve as a function of the wavelength, has many possibilities to select the wavelengths of the first and the second blue light and their associated intensities.

In an embodiment for melatonin suppression, the predominant first wavelength of the first blue light is selected in a range of 460 to 480 nm, thus near to the maximum of the melatonin suppression curve, which occurs at about 470 nm. Preferably, the predominant first wavelength is selected to coincide with this maximum. The second and third wavelengths of the second blue light are now selected on either side of the predominant first wavelength and thus at wavelengths at which the melatonin suppression is lower than maximum. Preferably, the second predominant wavelength is selected in a range of 430 to 450 nm, and the third predominant wavelength is selected in a range of 480 to 500 nm. These ranges are preferred because they have a different melatonin suppression effect and are within the non-zero part of the visual eye sensitivity curve.

In an embodiment, a first light source generates the first blue light, a second light source generates the light with the second predominant wavelength, and a third light source generates the light with the third predominant wavelength. Controlling three separate light sources is easier than changing the spectrum of one light source. Preferably, the light sources are LEDs. Alternatively, the light sources may be formed by suitably selected phosphors which are hit by electrons, such as in a CRT or PDP display apparatus.

In an embodiment, the control signal which controls whether the first blue light or the second blue light is generated is received by a wired or wireless link, for example via the Internet or telephone. This allows controlling the amount of melatonin suppression from a central point. The control signal may be linked to the time to synchronize the amount of melatonin suppression with the real day/night cycle. Alternatively, the amount of melatonin suppression may be controlled in accordance with an artificial day/night cycle for people who, for example, have to work in night shifts.

Alternatively, a light sensor may be used to control the amount of melatonin suppression. Preferably, this light sensor is positioned to receive outside light. Even if a person is working in an environment in which no or only a low amount of daylight enters, it is possible to synchronize the selection of the spectral composition of the blue light such that the melatonin suppression is linked to the real day/night cycle.

In an embodiment, the display device further comprises sensing means for generating the control signal in dependence on biofeedback from a user. Now, the actual biological state of the user is used to control the photo-biological effect. The sensing means may comprise at least one out of: a skin/rectal temperature sensor, eye blinking sensor, eye movement sensor, skin conductance sensor, or a user activity detector. Each one of these sensors senses a particular issue of the biological state of the user, and may be used separately or in any combination to control the photo-biological effect in a desired manner. The user-activity detector may be constructed for sensing a number of keystrokes per minute, or the intensity of the use of a mouse.

In one application, the first, second and third light sources, which all generate blue light, are incorporated in the pixels of a display apparatus. Preferably, these pixels further also have a red and a green light source such that the pixels are able to produce white light. In accordance with the invention, the blue primary of the display can have different spectral compositions such that different melatonin suppression effects are obtained but still substantially the same blue color and intensity is achieved. Consequently, the white color of the display is substantially independent of the actual melatonin suppression effect selected. Preferably, the light sources are narrow band or monochromatic, such as LEDs or lasers.

In another application, in a backlight unit for illuminating a display, the first, second and third light sources are combined with red and green light sources to obtain white light the color point of which is substantially independent of the blue light sources that are activated.

These and other aspects of the invention are apparent from and will be elucidated with reference to the embodiments described hereinafter.

### BRIEF DESCRIPTION OF THE DRAWINGS

In the drawings:

FIG. 1 shows the human eye sensitivity curves for the red, green and blue cones,

FIG. 2 shows x, y, z curves according to the CIE 1931 standard observer,

FIG. 3 shows the effect of a light source with a wavelength corresponding to the peak of the melatonin sensitivity curve,

FIG. 4 shows the combined effect of two light sources with wavelengths selected around the wavelength corresponding to the peak of the melatonin sensitivity curve,

FIG. 5 shows an embodiment of a display apparatus comprising an LCD panel and a backlight unit with LED light sources in accordance with the present invention,

FIG. 6 shows a CRT with phosphor light sources, and

FIG. 7 shows the CIE1931 chromaticity diagram.

It should be noted that items which have the same reference numbers in different Figures, have the same structural features and the same functions, or are the same signals. In cases where the function and/or structure of such an item has been explained, there is no necessity for repeated explanation thereof in the detailed description.

### DETAILED DESCRIPTION

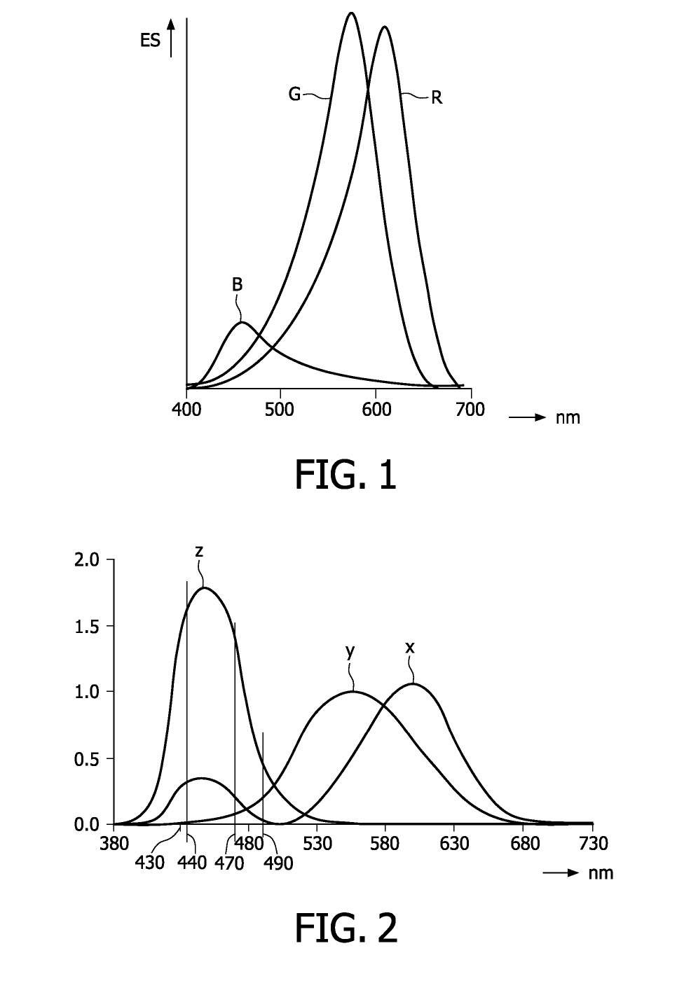

FIG. 1 shows the human eye sensitivity curves for the red, green and blue cones. The wavelength of the light is indicated along the horizontal axis in nm, the human eye sensitivity ES is indicated along the vertical axis. The human retina has three kinds of cones: cones which are sensitive to red light and which are referred to as red cones, cones which are sensitive to green light and which are referred to as green cones, and cones which are sensitive to blue light and which are referred to as blue cones. The response of the red cones as a function of the wavelength of the incident light is shown by the curve indicated by R. The response of the green cones as a function of the wavelength of the incident light is shown by the curve indicated by G. The response of the blue cones as a function of the wavelength of the incident light is shown by the curve indicated by B. The red cones have maximum sensitivity at 580 nm, the green cones have maximum sensitivity at 545 nm, and the blue cones have maximum sensitivity at 440 nm.

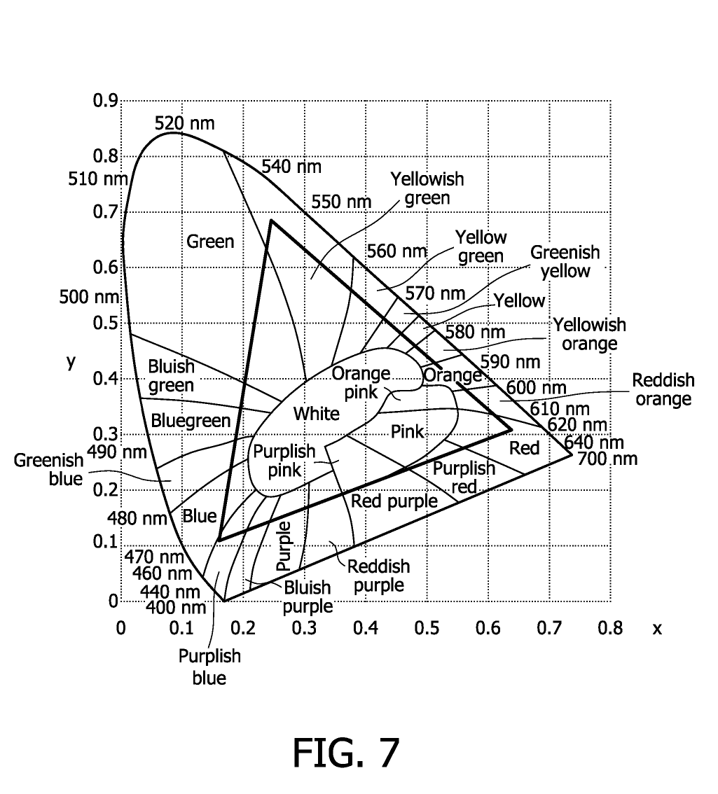

FIG. 2 shows x, y, z Standard Colorimetric Observer XYZ functions according to the CIE 1931 standard observer. FIG. 2 shows the color-matching functions as standardized by the CIE (ISO/CIE 10527: http://www.cie.co.at/publ/abst/10527.html; expected to be replaced soon by CIE Draft Standard DS 014-1.2/E:2004: http://www.cie.co.at/publ/abst/ds014_{—}1.pdf). The curves of FIG. 2 are used to calculate the x, y value of a light spectrum to locate a particular color within the CIE 1931 chromaticity diagram (see FIG. 7), using the formulas:

$$X = {\int }_{0}^{∞} I ( λ ) \overline{x} ( λ ) d λ$$

$$Y = {\int }_{0}^{∞} I ( λ ) \overline{y} ( λ ) d λ$$

$$Z = {\int }_{0}^{∞} I ( λ ) \overline{z} ( λ ) d λ$$

where I(λ) is the spectral power distribution (Watt/nm) of the light, λ is the wavelength of the light, and x(λ), y(λ) and z(λ) are the CIE 1931 Standard Colorimetric Observer XYZ functions. The CIE1931 chromaticity co-ordinates (x, y values) are calculated as:

$$x = \frac{X}{X + Y + Z}$$

$$and$$

$$y = \frac{Y}{X + Y + Z}$$

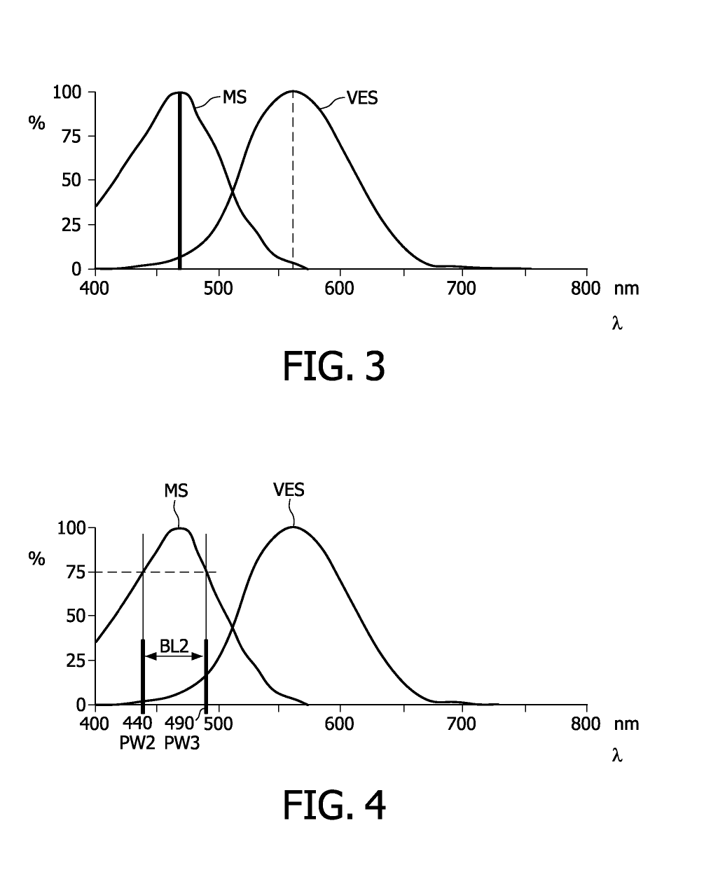

FIG. 3 shows the effect of a light source with a wavelength corresponding to the peak of the melatonin sensitivity curve. The melatonin suppression curve MS shows that the melatonin suppression effect has a maximum at 470 nm. The curve VES shows that the visual eye sensitivity has a maximum at about 560 nm. The visual eye sensitivity curve is determined by the three response curves R, G and B shown in FIG. 1.

By way of example, it is assumed that the blue light is generated with an intensity of 1 W/nm at a wavelength of 470 nm. Thus, this blue light has a wavelength which coincides with the maximum of the melatonin suppression effect, and has a normalized melatonin suppressing stimulus of 100%. The visual CIE1931 properties of this blue light are defined by:

x=0.1954/(0.1954+0.910+1.2876)=0.124

y=0.0910/(0.1954+0.910+1.2876)=0.058

Y=683*0.0910=62 lumen.

The CIE 1931 Standard Colorimetric Observer XYZ is a model to describe the color appearance of light as seen by the average human eye. Spots of light having the same x, y coordinates in the CIE 1931 chromaticity diagram and the same Y value are observed as identically colored spots of light, independent of the spectral composition of these spots of light.

FIG. 4 shows the combined effect of two light sources with wavelengths selected around the wavelength corresponding to the peak of the melatonin suppression curve. The same melatonin suppression curve MS and visual eye sensitivity curve VES as in FIG. 3 are shown. The first one of the light sources generates blue light having a wavelength of 440 nm and an intensity of 0.361 W/nm, the second one of the light sources generates blue light having a wavelength of 490 nm and an intensity of 0.397 W/nm.

The resulting combined blue light has a total normalized melatonin suppressing stimulus of 57%, and the visual properties are defined by

x=0.132

y=0.087

Y=683*0.0910=62 lumen

The melatonin suppressing stimulus is calculated by adding the contribution of the blue light at 440 nm, which is 0.361*0.75, to the contribution of the blue light at 490 nm, which is 0.397*0.77.

The values x, y, Y are calculated by adding together the contributions of the blue light at 440 nm and at 490 nm:

x=(0.3483*0.361+0.0320*0.397)/N

y=(0.0230*0.361+0.2080*0.397)/N

Y=683*y*N

N=(0.3483*0.361+0.0320*0.397)+(0.0230*0.361+0.2080*0.397)+(1.7471*0.361+0.4652*0.397)

From FIGS. 3 and 4 and the corresponding calculations it follows that a switch over between generating the blue light by a single source with a wavelength of 470 nm and generating the blue light by two sources with a wavelength of 440 nm and 490 nm, respectively, changes the melatonin suppressing stimulus from 100% to 57%, while the visual stimulus has substantially the same color (x, y changes from 0.124, 0.058 to 0.132, 0.087) and a substantially identical luminance (Y=62 lumen in both cases).

Thus, in the terminology used in the claims, the combination of the light with the predetermined second wavelength (440 nm in this embodiment) and the predetermined third wavelength (490 nm in this embodiment) has substantially the same color and intensity as the light with the predetermined first wavelength (470 nm in this embodiment). Preferably, the first wavelength is selected at, or around, the maximum of the melatonin suppression curve MS. For example, the first wavelength is selected in the range from 460 to 480 nm. The second wavelength is selected to be shorter than the first wavelength. Preferably, the second wavelength is selected in the range from 430 to 450 nm. The second wavelength should be selected within the non-zero part of the visual eye sensitivity curve VES. The third wavelength is selected to be longer than the first wavelength. Preferably, the third wavelength is selected in the range from 480 to 500 nm. The difference between the second and third wavelengths with respect to the first wavelength is determined by the desired difference in melatonin suppression effect. The intensity of the light with the second and third wavelengths is selected such that the combined intensity is substantially identical to the intensity of the light with the first wavelength. Further, the intensity of the light with the second and third wavelengths has to be selected such that the color of the light of the first wavelength and the color of the combined light of the second and the third wavelength are substantially the same. Small differences in color and/or luminance are allowable. Preferably, the observer does not see any noticeable differences between the different lights. To further boost the melatonin suppression effect without influencing the visual appearance, it is possible to add a light source with such a short wavelength that it is invisible but still within the non-zero part of the melatonin suppression curve MS.

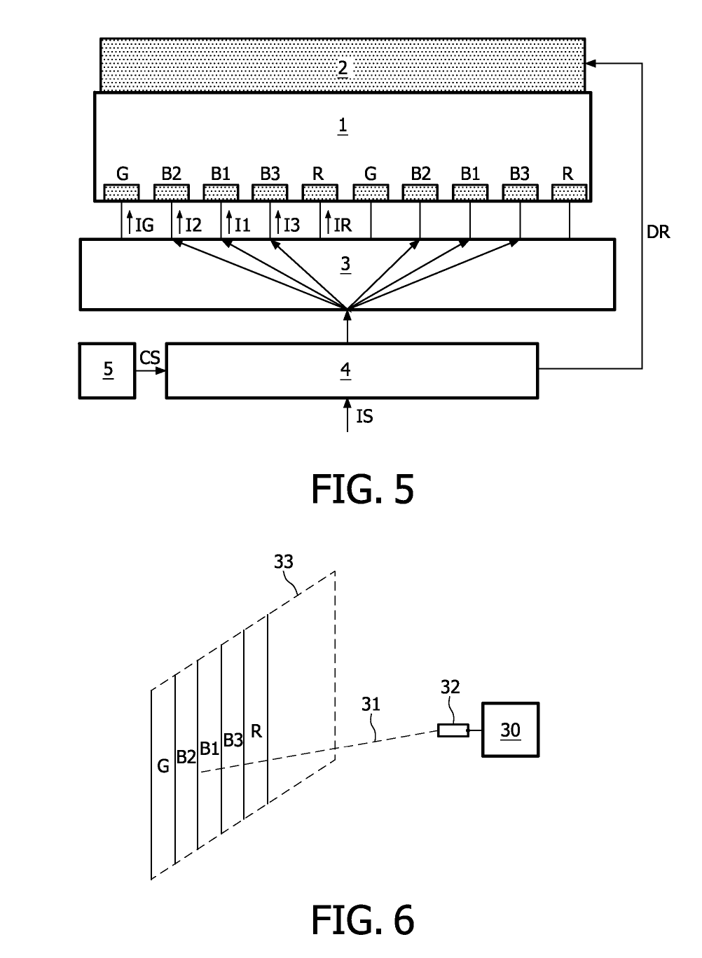

FIG. 5 shows an embodiment of a display apparatus comprising an LCD panel and a backlight unit with LED light sources in accordance with the present invention. The display apparatus comprises the backlight unit 1 which illuminates the LCD panel 2. The backlight unit 1 comprises an array of LEDs. The green LEDs G emit green light, the red LEDs R emit red light, the blue LEDs B1 emit blue light at a wavelength of predominantly 470 nm, the blue LEDs B2 emit blue light at a wavelength of predominantly 440 nm, and the blue LEDs B3 emit blue light at a wavelength of predominantly 490 nm. By a wavelength predominantly at a particular nm is meant that the LED emits light at only this wavelength, or in a small range around this wavelength, or that the intensity of the light has a maximum at this particular wavelength. As shown in FIG. 5, preferably, to optimize the spatial resolution, the blue LEDs B1 are positioned in between the blue LEDs B2 and B3.

A driver 3 supplies currents IG, IR, I1, I2, I3 to the green LEDs G, the red LEDs R, the blue LEDs B1, the blue LEDs B2, and the blue LEDs B3, respectively. A controller 4 controls the driver 3 to supply the currents IG, IR, I1, I2, I3 corresponding to a desired melatonin suppression effect, color and intensity.

The controller receives a control signal CS from a control signal generating circuit 5 which, for example, may comprise a time generator, a light sensitive element, or a trigger circuit.

The time generator generates the control signal CS for switching at predetermined switching instants between generating the blue light either by the LEDs B1 or by the combination of the LEDs B2 and B3. These switching instants may be synchronized with a real or artificial day/night cycle. Artificial day/night cycling may be interesting, for example, for people who have to work at night or who live in a situation where they are not exposed to the real day/night cycling. It is not required that, at the switching instants, the current through the LEDs B1 is switched off completely; it is possible to gradually dim the LEDs B1 while the brightness of the LEDs B2 and B3 is gradually increased. The same is true for a switch over from the LEDs B2 and B3 to the LEDs B1. This gradual switch over may occur within several hours.

The light sensitive element may be used to receive real daylight and to generate a control signal CS which controls the switch over in synchronism with the outside light conditions. This is especially interesting in situations where a person does not receive sufficient daylight, or receives predominantly light emitted by the display apparatus.

The trigger circuit may be coupled, wired or wirelessly (via telephone or the Internet), with a central system, usually a server, which controls the switch over and thereby the variation of the melatonin suppressing effect.

The control signal may also depend on a biophysical input or combinations thereof, such as, for example, body temperature, eye blinking frequency, computer keyboard/mouse use (for example intensity and/or speed of movement of the mouse) to measure fatigue and to adjust the color of the light to a desired alertness/sleepiness setting.

In a display apparatus, the controller 4 further receives an image signal IS which determines the image to be displayed on the LCD panel and supplies a drive signal DR to the LCD panel to modulate the transmission of the pixels in accordance with the image signal IS. Alternatively, if the LCD panel is not present and the matrix of LEDs forms the display panel, this image signal IS controls the currents through the LEDs G, R, B1 or G, R, B2, B3 in accordance with the image signal IS such that the image is displayed. In another alternative, in a display apparatus with a LCD panel an option exists to put the LCD panel in a predetermined transmission state (preferably maximum), which is not controlled anymore by the image signal IS, so that the display apparatus can be used as a bright light generator. Software may ask the user some questions, i.e. to provide advice on, or to determine, the duration of the exposure to the light, the variation of the blue spectrum over time, and the intensity of the light. If the bright light is used to cater for a time shift of the day/night cycle, for example due to air travel, the questions may relate to the current time at the departure location and the most recent wake up time. The software may require input on the current time at the destination location.

An example of the variation of the blue spectrum is given next. In the early morning, when the person starts working, for example at 8 o'clock, the light generated has a high melatonin suppression effect which gradually decreases to a minimum just before lunch. After lunch the melatonin suppressing effect is increased steeply and then decreases slowly again until the person leaves his workspace. Optionally, just before the person goes home the light may be changed to increase the melatonin suppressing effect, stimulating the alertness of the person during travel and reducing accident risk.

FIG. 6 shows a CRT with phosphor light sources. The display screen 33 of the CRT (Cathode Ray Tube) comprises phosphor stripes. The unit 32 comprises an electron gun for generating an electron beam 31 with a controllable intensity and a deflection unit for deflecting the electron beam 31 to the desired position on the screen 33. The phosphors emit light when hit by the electron beam 31. The color of light emitted by the phosphors is indicated by G for green, R for red, B1 for the first blue color, B2 for the second blue color, and B3 for the third blue color. Instead of phosphor stripes, phosphor dots may be used. Usually, in a color CRT with three different phosphors, three electron beams, one for each color phosphor, are generated which are separately controllable. In the display in accordance with the present invention, five separately controllable electron beams may be generated, of which 3 (R, G, B1) are active when maximum melatonin suppression is required or 4 (R, G, B2, B3) are active when minimum melatonin suppression is required. During a transition phase between these two states, 5 electron beams (R, G, B1, B2, B3) are active. In a PDP (Plasma Display Panel), the electrons are generated by an ignition of plasma. The controller 30 controls the intensity of the electron beam or beams 31 and the deflection thereof, as is well known from display apparatuses which comprise a CRT.

FIG. 7 shows the CIE1931 chromaticity diagram. In this well known diagram the X is depicted along the horizontal axis and the Y is depicted along the vertical axis. Because the Figure is in black and white, areas are indicated by their color's name. As is well known, although specific areas of colors are shown, these colors gradually change.

It should be noted that the above-mentioned embodiments illustrate rather than limit the invention, and that those skilled in the art will be able to design many alternative embodiments without departing from the scope of the appended claims.

For example, the LEDs of the backlight unit 1 may be OLEDs, lasers, gas discharge lamps, or fluorescent tubes, or any combination thereof. The discharge lamps may comprise different light sources in the same bulb. Instead of an LCD panel 2 in front of the backlight unit 1, any other display panel with a locally controllable transmission can be used. The present invention may be implemented in all apparatuses with a display device, such as, for example, television sets, computer monitors, PDAs, mobile phones, photo and film cameras.

Alternatively, the display unit may be absent altogether and the backlight unit is a lighting unit for generating so called “bright light therapy”.

It is not required that the different blue spectrums are exact metamerisms, slight deviations are allowed. If the light is used in a display it is possible to electronically correct for these deviations, for example by slightly adapting the currents through the LEDs.

Instead of a day/night synchronization of the blue spectrum, in special conditions other synchronizations are possible to control the biological rhythm. Such special conditions may be: traveling in boats, airplanes, spaceships, submarines; locations on earth near the poles where the light/dark cycles strongly change over the year; or people that work in night shifts.

In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. In the device claim enumerating several means, several of these means may be embodied by one and the same item of hardware. The mere fact that certain measures are recited in mutually different dependent claims does not indicate that a combination of these measures cannot be used to advantage.

1. A device for generating at least blue light and having a control circuit (4) for receiving a control signal (CS) defining a variation of a spectrum of the blue light to control a photo-biological effect of a vertebrate by generating first blue light (BL1) with a first predominant wavelength (PW1) having a first photo-biological effect, and/or second blue light (BL2) with both a second predominant wavelength (PW2), being shorter than the first predominant wavelength, and a third predominant wavelength (PW3), being longer than the first predominant wavelength, wherein the second blue light (BL2) has a second photo-biological effect smaller than the first photo-biological effect, while the first blue light (BL1) and the second blue light (BL2) have substantially identical colors and intensities.

2. A device as claimed in claim 1, wherein the first predominant wavelength (PW1) is selected in a range of 460 to 480 nm, the second predominant wavelength (PW2) is selected in a range of 430 to 450 nm, and the third predominant wavelength (PW3) is selected in a range of 480 to 500 nm.

3. A device as claimed in claim 1, further comprising a first light source (B1) for generating the first blue light (BL1), a second light source (B2) for generating blue light with the second predominant wavelength (PW2), and a third light source (B3) for generating blue light with the third predominant wavelength (PW3).

4. A device as claimed in claim 3, wherein the first light source (B1) comprises a first LED, the second light source (B2) comprises a second LED, and the third light source (B3) comprises a third LED, and the device further comprises a drive circuit (3) for generating a first current (I1) through the first LED, a second current (I2) through the second LED, and a third current (I3) through the third LED, and the control circuit (4) is constructed for controlling the driver (3) to generate the first current (I1), the second current (I2), and the third current (I3) to obtain light having a desired color, intensity, and a photo-biological effect.

5. A device as claimed in claim 3, wherein the first light source (B1) is a first type of phosphor, the second light source (B2) is a second type of phosphor, and the third light source (B3) is a third type of phosphor, and the device further comprises a drive circuit (30) for deflecting an electron beam (31) to either hit the first type of phosphor or both the second type of phosphor and the third type of phosphor.

6. A device as claimed in claim 1, wherein the control signal (CS) is a trigger signal obtained by a wired or wireless link.

7. A device as claimed in claim 1, further comprising a time generating circuit (5) for generating the control signal (CS) having a cyclical behaviour to control the photo-biological effect cyclically.

8. A device as claimed in claim 7, wherein the time generating circuit (5) is constructed for generating the control signal (CS) synchronized with the day/night cycle.

9. A device as claimed in claim 6, further comprising a light sensitive element (5) for generating the trigger signal (CS) in response to an amount of light impinging on the light sensitive element.

10. A display device comprising pixels, each comprising the first light source (B1), the second light source (B2) and the third light source (B3), as claimed in claim 3.

11. A display device as claimed in claim 10, wherein the pixels each further comprise a fourth light source (G) and a fifth light source (R) for enabling the pixels to emit white light.

12. A display device as claimed in claim 11, wherein the fourth light source (G) emits green light and the fifth light source (R) emits red light.

13. A display device as claimed in claim 11, wherein the first light source (B1) comprises a first LED, the second light source (B2) comprises a second LED, the third light source (B3) comprises a third LED, the fourth light source (G) comprises a fourth LED, and the fifth light source (R) comprises a fifth LED, and the display device further comprises a drive circuit (3), and the control circuit (4) is constructed for receiving the control signal (CS) and an image signal (IS) to control the driver (3) to generate a first current (I) through the first LED, a second current (I2) through the second LED, and a third current (I3) through the third LED, a fourth current (IG) through the fourth LED, and a fifth current (IR) through the fifth LED, to obtain light having a desired color and intensity in accordance with the image signal (IS), and a photo-biological effect.

14. A display device as claimed in claim 10, further comprising sensing means for generating the control signal depending on biofeedback from a user.

15. A display device as claimed in claim 14, wherein the sensing means comprises at least one out of: a skin/rectal temperature sensor, eye blinking sensor, eye movement sensor, skin conductance sensor, or a user-activity detector.

16. A display device as claimed in claim 15, wherein the user-activity detector is constructed for sensing a number of keystrokes per minute, or the intensity of the mouse use.

17. A display device as claimed in claim 1, wherein the photo-biological effect is a melatonin suppression effect or an alertness level.

18. A backlight unit for a display device comprising the first light source (B1), the second light source (B2) and the third light source (B3), as claimed in claim 3.

19. A method of generating at least blue light in response to a control signal (CS) defining a variation of a spectrum of the blue light to control a photo-biological effect of a vertebrate by generating first blue light (BL1) with a first predominant wavelength (PW1) having a first melatonin suppression effect, or second blue light (BL2) with both a second predominant wavelength (PW2), being shorter than the first predominant wavelength, and a third predominant wavelength (PW3), being longer than the first predominant wavelength, wherein the second blue light (BL2) has a second melatonin suppression effect smaller than the first melatonin suppression effect, while the first blue light (BL1) and the second blue light (BL2) have substantially identical colors and intensities.
